(19) United States
(12) Patent Application Publication
(10) Pub. No.: US 2025/0099062 A1
(43) Pub. Date: Mar. 27, 2025

(54) METHOD AND APPARATUS FOR ESTIMATING MATERIAL THICKNESSES IN RADIOLOGICAL PROJECTION IMAGES

(71) Applicant: Siemens Healthineers AG, Forchheim (DE)

(72) Inventors: Ferdinand LUECK, Erlangen (DE); Ludwig RITSCHL, Buttenheim (DE); Marcel BEISTER, Erlangen (DE); Steffen KAPPLER, Hallerndorf-Pautzfeld (DE)

(73) Assignee: Siemens Healthineers AG, Forchheim (DE)

(21) Appl. No.: 18/892,788

(22) Filed: Sep. 23, 2024

(30) Foreign Application Priority Data
Sep. 26, 2023 (EP) 23199686.9

(51) Int. Cl. A61B 6/00; A61B 6/50; G06T 7/00

(52) U.S. Cl. CPC A61B 6/5211; A61B 6/481; A61B 6/502; A61B 6/505; G06T 7/0014; G06T 2207/10116; G06T 2207/30068

(57) ABSTRACT

One or more example embodiments relates to a method for estimating material thicknesses in radiological projection images, comprising the steps:
providing a plurality of N projection images at different recording energies in each case,
performing a first decomposition of the N projection images into N thickness maps, which in each case represent the thickness of N regions each with different materials,
performing a second decomposition of a number of main thickness maps based on the N thickness maps and/or on corresponding measurements and a number of the N projection images into N result thickness maps, which in each case represent the thickness of the N regions each with different materials.

One or more example embodiments further comprises an apparatus and an imaging device.

### CROSS-REFERENCE TO RELATED APPLICATION(S)

The present application claims priority under 35 U.S.C. § 119 to European Patent Application No. 23199686.9, filed Sep. 26, 2023, the entire contents of which are incorporated herein by reference.

### FIELD

One or more example embodiments relates to a method and an apparatus for estimating material thicknesses in radiological projection images and an imaging device. For example, one or more example embodiments relates to projection-based multi-energy decomposition.

### RELATED ART

In spectral imaging, a so-called “dual-energy approach” is often used to visualize the concentration of iodine or bone, for example. Two recordings at different energies can be used to estimate values for two different materials or the thickness of different regions. For example, in mammography, it is assumed that the thickness of the breast is constant and this can be used to separate glandular tissue and iodine concentration.

However, after compression of the breast, there are often regions of different thickness, for example the edge of the breast, skin folds in the armpit region or the breast itself, which are caused by an inclination of the paddle from the chest wall to the standing side. If there is an iodine concentration in such a region, it is not possible to distinguish whether this is a change in thickness or a real iodine concentration.

Moreover, radiography also includes the aim of separating bone and water. However, the presence of fat or other materials leads to incomplete decomposition and leaves bone artifacts in the “water” image.

Currently, contrast-enhanced mammography is performed using low- and high-energy recording with a weighted logarithmic subtraction algorithm to visualize the iodine concentration. This leads to regions of different thickness resulting in an inhomogeneous background intensity after logarithmic subtraction. Several post-processing algorithms are available for attempting to compensate this inhomogeneity while at the same time maintaining the iodine concentrations in these regions.

In radiography, the principle is the same, but incomplete subtraction leads to visible bone structures in the “water” image. No correction is currently applied in this respect.

### SUMMARY

One or more example embodiments provide a method and an apparatus for estimating material thicknesses in radiological projection images and an imaging device with which the above-described drawbacks are avoided.

### BRIEF DESCRIPTION OF THE DRAWINGS

One or more example embodiments is described in detail below with reference to the appended figures. Herein, the same components are provided with identical reference symbols in the different figures. The figures are generally not to scale. In the figures:

FIG. 1 shows a radiography system with an apparatus according to one or more example embodiments,

FIG. 2 shows a graph with attenuation levels and recording energies, and

FIG. 3 shows an example of a method for estimating material thicknesses in radiological projection images according to one or more example embodiments.

### DETAILED DESCRIPTION

A method according to one or more example embodiments is used to estimate material thicknesses in radiological projection images. It comprises the following steps:
providing (in particular recording or loading from a storage unit) a plurality of N projection images at different recording energies in each case,
performing a first decomposition of the N projection images into N thickness maps, which in each case represent the thickness of N regions each with different materials,
performing a second decomposition of a number of main thickness maps based on the N thickness maps and/or on corresponding measurements and a number of the N projection images into N result thickness maps, which in each case represent the thickness of the N regions each with different materials.

The provision of projection images is sufficiently well known. For example, the N projection images can be recorded with a dual or multi-energy X-ray device and decomposed directly with the method. However, the projection images may have been recorded earlier and then stored. In this case, provision takes the form of downloading from a data memory. The projection images can be recorded simultaneously or successively. The only important thing is that they show the same subject in the same position. It is theoretically also possible to correct motion artifacts, for example by image registration.

Basically, the only thing that is important for the method is that two or more projection images are available that show the same scene at different energies. Even if the method is already advantageous at two different energies, an advantage becomes even more apparent at three or more energies. To help to understand the method, it can be imagined that three projection images are recorded: an H projection image PH at a highest recording energy, an M projection image PM at a middle recording energy and an L projection image PL at a lowest recording energy.

It is advantageous if the recording energies are selected such that the projection images do not overlap in terms of energy (i.e., the same energies are mapped in two images) and the energies lie in regions in which in each case at least one material differs from the other materials with regard to attenuation (this also refers to the attenuation of X-rays). With regard to a contrast agent, a recording energy can, for example, be selected such that it lies in the K-edge of the contrast agent.

In the course of one or more example embodiments, not just one but two decompositions are performed in which N thickness maps are compiled from the projection images (and, in the second, also from main thickness maps). Herein, the thickness maps in each case represent the thickness of N regions each with different materials. The thickness maps basically correspond to the projection images with the difference that their “image points” do not show attenuation levels in the form of gray tones, but in the form of thickness information, for example in millimeters. Together, the thickness maps generally give the total thickness of the breast at the corresponding “image points”.

In the first decomposition, the N projection images are decomposed into N thickness maps. For this purpose, a P vector from the N projection images can, for example, be calculated with an N×N matrix to form the N thickness maps. It should be noted that the projection images are formed from image points. Herein, each point at the location (x,y) of a thickness map is formed from the corresponding image points at the location (x,y) of projection images. For example, the matrix M* and the p vector (P1(x,y), P2(x,y), . . . , PN(x,y)) at the corresponding coordinates (x,y) can be used to calculate the thickness images point-by-point in the d vector (d1(x,y), d2(x,y), . . . , dN(x,y)) where d=M*·P. This decomposition is described in more detail below.

The first decomposition is followed by the second decomposition. In contrast to the first decomposition, not only projection images, but also at least one main thickness map D are included in the calculation. Therefore, for example, a vector (D, P1+P2, P3, . . . , PN) is formed as an input vector and multiplied by the matrix K*.

The matrices M* and K* are simple to specify. They are preferably calculated as inverse matrices from M and K, which are formed from attenuation coefficients for the materials in question. This is explained in more detail below.

The main thickness map D can be based directly on the N thickness maps, i.e., for example D=d1+d2+ . . . +dN, however it can also be based on corresponding measurements, for example by measuring the recorded breast directly or by using a model that is measured. Here, it should be noted that D basically indicates the total thickness of the breast. Therefore, D(x,y) is a map that indicates the total thickness of the breast at the positions (x,y).

It would now be possible to simply add the thickness map to the N projection images and calculate a (N+1)×N vector with a N×(N+1) matrix as the input vector. However, as will be shown below, it is advantageous to calculate the matrix (K*) as an inverse matrix from a matrix K in which attenuation coefficients are used. Therefore, quadratic matrices are suitable. For this purpose, one projection image for each main thickness map can be omitted from the N projection images, for example one that only makes a small contribution. However, in order to be able to work with as much information as possible, it is advantageous for two or more projection images to be combined. This is described in more detail below.

The result thickness maps can then be output, for example directly on a display for evaluation, or they can be stored in a data memory.

An apparatus according to one or more example embodiments is used to estimate material thicknesses in radiological projection images. It is preferably configured to perform the method according to one or more example embodiments and comprises the following components:
a data interface for receiving a plurality of N projection images at different recording energies in each case, preferably an H projection image recorded at a highest recording energy (for example of three), an M projection image recorded at a middle recording energy (for example of three) and an L projection image recorded at a lowest recording energy (for example of three),
a decomposition unit configured for:
i) a first decomposition of the N projection images into N thickness maps, which in each case represent the thickness of N regions each with different materials, and
ii) a second decomposition of a number of main thickness maps, based on the N thickness maps and/or on corresponding measurements and a number of the N projection images, into N result thickness maps, which in each case represent the thickness of the N regions each with different materials, preferably for decomposing three projection images into three thickness maps, which in each case represent the thickness of three different materials.

The functions of the components of the apparatus have already been described in the course of the method.

An imaging device according to one or more example embodiments is preferably a multilayer detector or photon-counting detector. It is configured for spectral recording of an object via radiation and comprises a radiation source configured for emitting a spectrum comprising N different recording energies, a detector unit configured to detect the N recording energies and an apparatus according to one or more example embodiments.

There are several options for recording a plurality of energies. The radiation source can emit a broadband spectrum that contains all desired recording energies. However, it can also emit individual (narrow-band) spectra for each recording energy or for sub-groups of recording energies (simultaneously or successively).

There are also several alternatives for the detectors. For example, a single detector can be used that is sensitive to all recording energies. In the case of a broadband radiation source, filters should be used that only allow one recording energy to pass through for recording a projection image in each case. Alternatively, the radiation source can emit successive narrow-band spectra for one recording energy in each case.

In another example, it is possible to use an energy-selective detector or a plurality of detectors which are only active in a narrow energy band in each case. Here, a plurality of recordings can be taken simultaneously at different recording energies.

One or more example embodiments can in particular be implemented in the form of a computer unit with suitable software. For this purpose, the computer unit can, for example, have one or more interacting microprocessors or the like. In particular, they can be implemented in the form of suitable software program parts in the computer unit. A largely software-based implementation has the advantage that previously used computer units can be easily retrofitted via a software or firmware update in order to operate in the manner according to one or more example embodiments. In this respect, the object is also achieved by a corresponding computer program product with a computer program that can be loaded directly into a memory facility of a computer unit, with program sections for executing all the steps of the method according to one or more example embodiments when the program is executed in the computer unit. In addition to the computer program, such a computer program product may comprise additional items, such as, for example, documentation and/or additional components, including hardware components, such as, for example, hardware keys (dongles etc.) for using the software.

A computer-readable medium, for example a memory stick, a hard disk or another kind of transportable or permanently installed data carrier, on which the program sections of the computer program that can be read in and executed by the computer unit are stored, can be used for transport to the computer unit and/or for storage on or in the computer unit.

Further particularly advantageous embodiments and developments emerge from the dependent claims and the following description, wherein the claims of one claim category can also be developed analogously to the claims and descriptive parts for another claim category and, in particular, individual features of different exemplary embodiments or variants can also be combined to form new exemplary embodiments or variants.

Preferably, the projection images P are decomposed into thickness maps d with a prespecified N×N matrix. This has already been explained above. For example, for the first decomposition, a P vector from the projection images can be multiplied by a suitable N×N matrix M* or, for the second decomposition, a PD vector formed from the projection images and at least one main thickness map can be multiplied by a suitable N×N matrix K*. Preferably, the matrix (for the first and/or the second decomposition) is an inverse matrix to a matrix formed from attenuation coefficients μij of the materials for the different energies.

In the preferred case, in which recording was carried out with (at least) three recording energies
an H projection image is provided, which was recorded at a highest recording energy,
an M projection image is provided, which was recorded at a middle recording energy and
an L projection image is provided, which was recorded at a lowest recording energy.

These three projection images are then decomposed into three (or possibly more) thickness maps during the first decomposition and, during the second decomposition, together with at least one main thickness map, also decomposed into three (or possibly correspondingly more) thickness maps, namely the result thickness maps.

This is explained in more detail below with reference to a preferred example in which an H projection image PH, an M projection image PM and an L projection image PL have been recorded with three different recording energies. This example can be easily extended to N energies or reduced to two energies using the principle described above.

The respective attenuation coefficients μ are known for different materials, as will be explained in more detail in the course of the description of FIG. 2. Thus, the attenuation coefficients μi (i=1, 2, 3) for the three materials at the energies H, M and L are known and can be specified as coefficients for a matrix M:

$$M = ( \begin{matrix}{μ}_{1}^{H} & {μ}_{2}^{H} & {μ}_{3}^{H} \\ {μ}_{1}^{M} & {μ}_{2}^{M} & {μ}_{3}^{M} \\ {μ}_{1}^{L} & {μ}_{2}^{L} & {μ}_{3}^{L}\end{matrix} ) \qquad ( 1 )$$

The following applies for the P vector (PH, PM, PL) and the d vector (d1, d2, d3): P=M·d, i.e., the respective thicknesses multiplied by the appropriate attenuation coefficients in the matrix result in the expected image values of the projection images. If the thickness of the respective materials were known everywhere (these would be the thickness maps), the projection images could be calculated.

However, since the projection images are known and the thickness maps are to be ascertained, it is advantageous to invert the matrix M to obtain the matrix M*. This is then followed by the coefficients mij, which can be calculated by inverting the matrix M shown above in (1):

$$( \begin{matrix}{d}_{1} \\ {d}_{2} \\ {d}_{3}\end{matrix} ) = ( \begin{matrix}{m}_{11} & {m}_{12} & {m}_{13} \\ {m}_{21} & {m}_{22} & {m}_{23} \\ {m}_{31} & {m}_{32} & {m}_{33}\end{matrix} ) ( \begin{matrix}{P}_{H} \\ {P}_{M} \\ {P}_{L}\end{matrix} ) \qquad ( 2 )$$

Thus, it is then possible to calculate three thickness maps d1, d2, d3 from the H projection image PH, the M projection image PM and the L projection image PL, i.e., to decompose the projection images into thickness maps.

As mentioned, it is additionally possible for further projection images that have been recorded with other energies to be provided. For each different energy, a thickness map can be provided for a further material or the previous results can be improved.

The procedure for the second decomposition is slightly different, since at least one main thickness map is used there. The following example is based on the previous calculation and the main thickness map D=d1+d2+d3 is formed. It would also be possible for the thickness D(x,y) to be measured during the recording or for the main thickness map to be derived from a model. However, the calculation of D described in the introduction is particularly advantageous, since no information other than the projection images needs to be available. Since it is still intended to use three coefficients, two projection images, namely the M projection image and the L projection image, are combined to form an ML projection image PML. With the matrix:

$$K = ( \begin{matrix}1 & 1 & 1 \\ {μ}_{1}^{M L} & {μ}_{2}^{M L} & {μ}_{3}^{M L} \\ {μ}_{1}^{H} & {μ}_{2}^{H} & {μ}_{3}^{H}\end{matrix} ) \qquad ( 3 )$$

the following applies for the PD vector (D, PML, PH) and the final df vector (df1, df2, df3): PD=K·df. Here, once again, the matrix K should be inverted to obtain the matrix K* to calculate the final thickness maps. This is then followed by the coefficients kij, which are calculated from the inversion of the matrix K above in (3):

$$( \begin{matrix}d {f}_{1} \\ d {f}_{2} \\ d {f}_{3}\end{matrix} ) = ( \begin{matrix}{k}_{11} & {k}_{12} & {k}_{13} \\ {k}_{21} & {k}_{22} & {k}_{23} \\ {k}_{31} & {k}_{32} & {k}_{33}\end{matrix} ) ( \begin{matrix}D \\ {P}_{M L} \\ {P}_{H}\end{matrix} ) . \qquad ( 4 )$$

Preferably, in one embodiment of the method, one of the thickness maps is
a K thickness map representing the thickness of contrast agent regions, and/or
a G thickness map representing the thickness of glandular tissue, and/or
an A thickness map representing the thickness of adipose tissue, and/or
a B thickness map representing the thickness of bone, and/or
a W thickness map representing the thickness of regions with water.

Preferably, in particular for a method with three recording energies, one of three thickness maps is a K thickness map, a another is a G thickness map, and another is an A thickness map.

Preferably, the recording energies are based on the attenuation properties of the materials underlying the thickness maps. As stated above, the recording energies should be selected from energy ranges in which the attenuation coefficient of at least one of the materials differs significantly from the others. Preferably, beam energies are selected at which differences in the attenuation properties of all materials, or of at least one material, to the others are maximum in each case. Preferably, a recording energy (during recording of the projection images) was selected such that it lies in the falling part of an absorption edge of one of the materials.

The lowest recording energy is preferably between 10 keV and 100 keV. A middle recording energy is preferably between 20 keV and 130 keV. The highest recording energy is preferably between 30 keV and 150 keV. However, the recording energies are strongly dependent on the materials to be decomposed. Care should be taken to ensure that, overall, energies are selected that enable good energetic separation of the desired materials.

According to a preferred method, the first decomposition of the projection images into thickness maps is performed multiple times and the thickness maps are used to correct beam hardening of the decomposed projection images and/or coefficients of a matrix used in the decomposition. This multiple performance of the first decomposition and correction takes place before the second decomposition, which only has to be performed once, but can also be carried out multiple times for a corresponding correction.

It should be noted here that different recording energies were used and that different materials absorb differently at different energies. This is compensated by correcting beam hardening. Basically, (pseudo-) monoenergetic attenuation is calculated from polyenergetic attenuation.

Preferably, herein, polynomial correction is applied. Herein, a corrected projection image Pkorr(x,y) can be created from a projection image P(x,y) via coefficients ca that can be ascertained or calculated for each image point (x,y) and from a previously defined degree A of the polynomial according to the following formula:

$${P}_{k o r r} ( x , y ) = {\sum }_{a}^{A} {c}_{a} {P}^{a} ( x , y ) \qquad ( 5 )$$

i.e., basically, the polynomial c0+c1P+c2P2+ . . . +CNPN. In this respect, it should be noted that the coefficients c basically also depend on the thickness maps, i.e., on the thickness of the materials through which a beam has to pass before striking an image point of the detector. Therefore, this means that the coefficients ca basically depend on (d1, d2, . . . , dN) for each image point. Here, it is quite feasible for this dependence to be negligible for some materials. If, for example glandular, adipose and iodine thickness maps are generated, it has been shown that a dependence on the main thickness map D (addition of the three thickness maps) and the glandular G thickness map, possibly together with the iodine thickness map, is sufficient. Then, preferably, ca (dg/D, D) or ca (dj/D, dg/D, D) would be assumed. This can be calculated or measured for different values for dg, dj and D.

In this regard, it should be noted that no thickness maps are available for the images recorded at the start of the method (“initial images”). Here, beam-hardening correction could initially be dispensed with and correction only performed on the next iteration. However, preferably, the initial images are also corrected and, to be precise, with prespecified thickness maps. For this purpose, in particular a uniform glandular tissue content of 50% is applied to the entire image for a breast recording.

As indicated above, preferably, in one embodiment of the method, a main thickness map D is formed by adding the (for example three) thickness maps. This is preferably used to correct beam hardening of the decomposed projection images and/or to form final thickness maps.

It is preferable for the first decomposition to be performed with the individual N projection images and the second decomposition to be performed with fewer than N projection images. It is then in particular possible to work with N×N matrices in each case.

Preferably, for the second decomposition, a plurality, in particular two, of the projection images are combined to form a unified V projection image, for example the above-described ML projection image PML.

Preferably, a main thickness map from an addition of the N thickness maps of the first decomposition is used for the second decomposition. Alternatively or additionally, a main thickness map from a model of the recorded object can be used for the second decomposition. Alternatively or additionally, a main thickness map from measurements of the recorded object can be used for the second decomposition.

Preferably, in one embodiment of the method, energetically adjacent projection images are combined to form a V projection image, however this is not mandatory. Herein, “energetically adjacent” means that the recording energies are directly adjacent to one another. In the preferred case of three recording energies, the M projection image PM and the L projection image PL are combined to form an ML projection image PML.

This combination preferably takes place via a weighted averaging of the image values in dependence on the intensity of the irradiation with the respective recording energy. For this purpose, preferably, image values at the same locations are added together and then normalized via a sum of the respective intensities. Basically, such weighted averaging is known from the prior art.

Preferably, in one embodiment of the method, for a matrix for the second decomposition, in addition, the attenuation coefficients that are combined are those which correspond to the recording energies of the combined projection images, preferably via weighted averaging. As explained above, in the case of three recording energies and the combination of the M projection image and the L projection image to form an ML projection image, the coefficients μM and μL are combined to form μML. Herein, preferably, in the case of three recording energies, the coefficients μML are calculated from weighted averaging of the coefficients μM and μL for the respective material in dependence on the intensity of the irradiation with the respective recording energy.

Preferably, in one embodiment of the method, a number of thickness maps, and/or a main thickness map, are additionally denoised. This preferably takes place via a method which takes into account a variable thickness of a recorded object and in particular assumes a continuous change in thickness. Herein, denoising preferably only takes place in the region of the (recorded) object.

A preferred apparatus comprises a correction unit configured to correct projection images and/or attenuation coefficients, in particular to correct beam hardening.

A preferred apparatus comprises a denoising unit configured to denoise thickness maps.

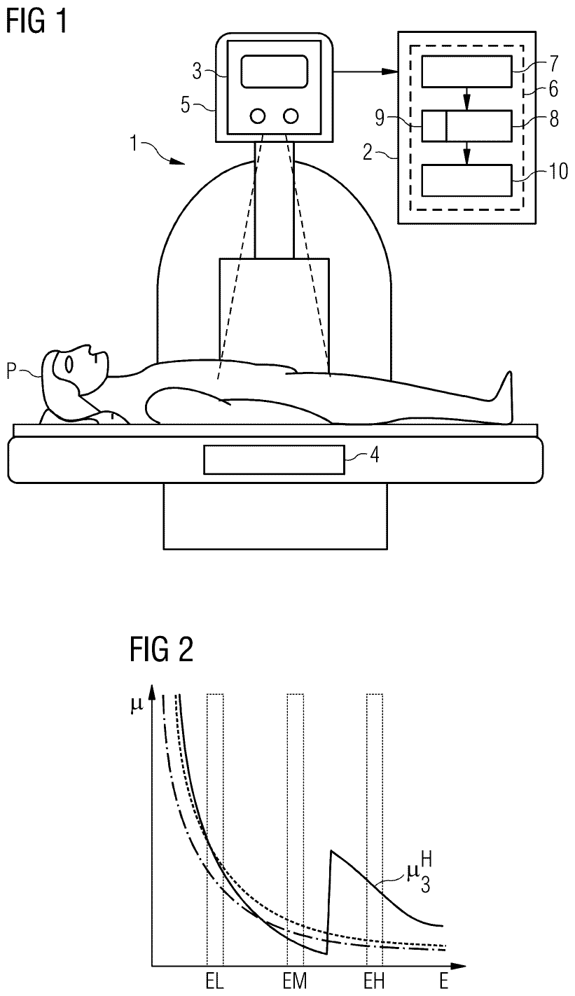

FIG. 1 shows, in a roughly schematic manner, an imaging device 1 in the form of an X-ray system 1 with a control facility 2. This is equipped with an apparatus 6 configured to perform the method according to one or more example embodiments. The X-ray system 1 usually has a radiation source 3, here representing an X-ray source, and during a recording of a projection image irradiates a patient P with a beam collimated by the collimator 5, so that the radiation strikes a detector 4 opposite the radiation source 3.

The only components of the control facility 2 depicted are those essential for explaining one or more example embodiments. Radiography systems 1 and associated control facilities 2 are in principle known to the person skilled in the art and therefore do not need to be explained in detail.

The control facility 2 comprises an apparatus 6 for estimating material thicknesses in radiological projection images. The apparatus 6 comprises a data interface 7, a decomposition unit 8, a correction unit 9 and a denoising unit 10.

The data interface 7 is used to receive a plurality of N projection images at different recording energies in each case, preferably an H projection image, which was recorded at a highest of three recording energies, an M projection image, which was recorded at a middle one of three recording energies and a L projection image, which was recorded at a lowest of three recording energies (see also FIG. 4).

The decomposition unit 8 is used to perform two successive decompositions, namely a first decomposition of the N projection images into N thickness maps, which in each case represent the thickness of N regions each with different materials, and a second decomposition of a number of main thickness maps based on the N thickness maps and/or on corresponding measurements and a number of the N projection images, into N result thickness maps, which in each case represent the thickness of the N regions each with different materials.

The correction unit 9 is configured to correct projection images. Herein, the first decomposition of the projection images into thickness maps can be performed multiple times and the thickness maps are used to correct beam hardening of the decomposed projection images. This can take place iteratively so that the correction is improved with the currently ascertained thickness maps in each case, which are based on the decomposition of improved projection images.

The denoising unit 10 is configured to denoise thickness maps. These can be the thickness maps obtained by the first decomposition or a main thickness map. The denoising is preferably carried out via a method that takes into account a variable thickness of a recorded object and in particular assumes a continuous change in thickness, wherein the denoising preferably only takes place in the region of the object.

FIG. 2 shows a graph with attenuation levels for X-rays and three recording energies EH, EM, EL. In the graph, the solid line shows, for example, the attenuation of iodine, the dashed line shows glandular attenuation and the dash-dotted line shows adipose attenuation. The K-edge is clearly identifiable in the attenuation of iodine. Projection images are now recorded at three (narrow) recording energies EH, EM, EL. At the lowest recording energy EL, the attenuation levels of iodine and glandular attenuation are almost the same and adipose attenuation differs from both of them. At the middle recording energy EH, the three attenuation levels are clearly different. The highest recording energy EH is the K-edge of iodine. Therefore, this is where the attenuation of iodine differs significantly from the other two.

If iodine were selected as a material 3, the attenuation coefficient μ could be selected from the line for iodine, for example, for a matrix for decomposition. In this example, it is indicated that with regard to FIG. 4 (with iodine as the third material), the coefficient μ3H can be selected there.

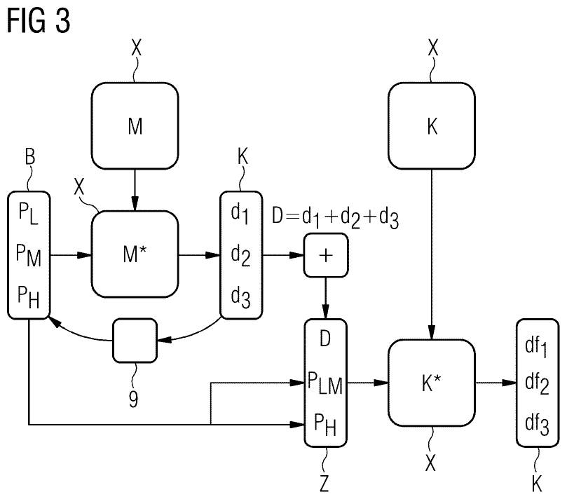

FIG. 3 shows a method for estimating material thicknesses in radiological projection images B. On the far left, it starts with the P vector (PL, PM, PH). These are three projection images B with image values at the coordinates (x,y) that have been provided. Although the following refers to the images B, it can be imagined that all calculations are performed with their image points.

PH denotes an H projection image, which was recorded at a highest of three recording energies, PM denotes an M projection image, which was recorded at a middle recording energy and PL denotes an L projection image, which was recorded at a lowest recording energy.

The M matrix X is now created from attenuation coefficients μ that were taken from FIG. 2 and at the same time the K matrix X is created with a (weighted) combination of the coefficients μM and μL to give μML. The two matrices X are then:

$$M = ( \begin{matrix}{μ}_{1}^{H} & {μ}_{2}^{H} & {μ}_{3}^{H} \\ {μ}_{1}^{M} & {μ}_{2}^{M} & {μ}_{3}^{M} \\ {μ}_{1}^{L} & {μ}_{2}^{L} & {μ}_{3}^{L}\end{matrix} ) and K = ( \begin{matrix}1 & 1 & 1 \\ {μ}_{1}^{M L} & {μ}_{2}^{M L} & {μ}_{3}^{M L} \\ {μ}_{1}^{H} & {μ}_{2}^{H} & {μ}_{3}^{H}\end{matrix} ) .$$

These two matrices X are inverted to obtain the matrices M* or K* and the maps K (the d vector (d1, d2, d3)) are first calculated from the P vector and the matrix M* where d=M*. P.

The thickness maps (d1, d2, d3) of the d vector can now be used to correct the projection images, possibly with a main thickness map D=d1+d2+d3. This takes place, for example with the formula Pkorr=c0(d2/D)+c1(d2/D) P+c2(d2/D)P2+c3(d2/D)P3. The corrected values Pkorr are then the new values for the P vector from which the maps K are ascertained again.

The second decomposition is now carried out with intermediate values Z, namely the main thickness map D, the H projection image PH and a V projection image PML, which is a combination of the L projection image PL and the M projection image PM. The PD vector indicated by the intermediate values Z is now multiplied by the matrix K*, resulting in final thickness maps df1, df2 and df3 as maps K with df=K*·PD.

Finally, it is pointed out once again that embodiments described above in detail merely refers to exemplary embodiments which can be modified by the person skilled in the art in a wide variety of ways without departing from the scope of the invention. Furthermore, the use of the indefinite articles “a” or “an” does not preclude the possibility that the features in question may also be present on a multiple basis. Likewise, terms such as “unit” do not preclude the possibility that the components in question may consist of a plurality interacting sub-components which may also be spatially distributed. The term “a number” should be understood as meaning “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for estimating material thicknesses in radiological projection images, the method comprising:
providing a plurality of N projection images at different recording energies;
performing a first decomposition of the N projection images into N thickness maps, the N thickness maps represent a thickness of N regions, respectively, each of the N regions with different materials; and
performing a second decomposition of a number of main thickness maps based on at least one of the N thickness maps or corresponding measurements or a number of the N projection images into N result thickness maps.

2. The method of claim 1, wherein the N projection images are decomposed into thickness maps with a prespecified N×N matrix.

3. The method of claim 1, wherein at least one of the thickness maps is
a K thickness map representing a thickness of contrast agent regions,
a G thickness map representing a thickness of glandular tissue,
an A thickness map representing a thickness of adipose tissue,
a B thickness map representing a thickness of bone, or a W thickness map representing a thickness of regions with water.

4. The method of claim 1, wherein the recording energies are based on attenuation properties of the materials underlying the N thickness maps.

5. The method of claim 1, wherein the performing the first decomposition of the N projection images into thickness maps is performed multiple times and the N thickness maps are used to correct at least one of beam hardening of the decomposed projection images or coefficients of a matrix used in the decomposition.

6. The method of claim 1, wherein a main thickness map is formed by adding the N thickness maps.

7. The method of claim 1, wherein the performing the first decomposition is performed with the N projection images and the performing the second decomposition is performed with fewer than N projection images.

8. The method of claim 7, wherein energetically adjacent projection images are combined to form a V projection image.

9. The method of claim 8, wherein, for a matrix for the second decomposition, in addition, attenuation coefficients that are combined are those which correspond to the recording energies of the combined projection images.

10. The method of claim 1, wherein at least one of a number of thickness maps or a main thickness map are denoised.

11. An apparatus configured to estimate material thicknesses in radiological projection images, the apparatus comprising:
a data interface configured to receive a plurality of N projection images at different recording energies; and
a decomposition unit configured to
perform a first decomposition of the N projection images into N thickness maps, which in each case represent the thickness of N regions each with different materials, and
perform a second decomposition of a number of main thickness maps based on at least one of the N thickness maps or on corresponding measurements and a number of the N projection images into N result thickness maps, the N thickness maps represent a thickness of the N regions with different materials.

12. An apparatus of claim 11, comprising at least one of:
a correction unit configured to correct at least one of projection images or attenuation coefficients, or
a denoising unit configured to denoise thickness maps.

13. An imaging device configured to spectral record an object via radiation, the imaging device comprising:
a radiation source configured to emit a spectrum comprising N different recording energies;
a detector unit configured to detect the N recording energies; and
the apparatus of claim 11.

14. A non-transitory computer program product comprising instructions which, when executed by a computer, cause the computer to perform the method of claim 1.

15. A non-transitory computer-readable storage medium comprising instructions which, when executed by a computer, cause the computer to perform the method of claim 1.

16. The method of claim 2, wherein the N×N matrix is an inverse matrix to a matrix formed from attenuation coefficients of the materials for the different energies.

17. The method of claim 16, wherein
an H projection image is provided, the H projection image recorded at a highest of three recording energies,
an M projection image is provided, the M projection image recorded at a middle one of three recording energies,
an L projection image is provided, the L projection image recorded at a lowest of three recording energies, and
the H projection image, the M projection image and the L projection image are decomposed into three thickness maps.

18. The method of claim 4, wherein beam energies are selected at which differences in the attenuation properties of all materials, or at least one material, to the others are a maximum.

19. The method of claim 18, wherein a lowest recording energy is between 10 keV and 100 keV, a middle recording energy is between 20 keV and 130 keV and a highest recording energy is between 30 keV and 150 keV.

20. The method of claim 6, wherein the main thickness map is used to at least one of correct beam hardening of the decomposed projection images or form final thickness maps.

21. The apparatus of claim 11, wherein the N projection images include an H projection image recorded at a highest of three recording energies, an M projection image recorded at a middle one of three recording energies, and an L projection image recorded at a lowest of three recording energies.
